(19) United States
(12) Patent Application Publication
(10) Pub. No.: US 2026/0301440 A1
(43) Pub. Date: Oct. 1, 2026

(54) SYSTEM AND METHOD FOR PREDICTING BLASTOCYST DEVELOPMENT USING FEATURE ENGINEERING

(71) Applicant: FUTURE FERTILITY INC., Toronto (CA)

(72) Inventors: Alexandr KRIVOI, Toronto (CA); Jullin FJELDSTAD, Toronto (CA); Weikai QI, Toronto (CA)

(21) Appl. No.: 19/478,071

(22) PCT Filed: Apr. 19, 2024
(86) PCT No.: PCT/CA2024/050521

§ 371 Date: Oct. 23, 2025

Related U.S. Application Data
(60) Provisional application No. 63/461,841, filed on Apr. 25, 2023.

(51) Int. Cl. G06V 20/69; G06N 3/0464; G06T 7/11; G06T 7/62; G16H 30/40

(52) U.S. Cl. CPC G06V 20/698; G06N 3/0464; G06T 7/11; G06T 7/62; G06V 20/695; G16H 30/40

(57) ABSTRACT

Methods and systems for predicting a quality of an oocyte based on an image of the oocyte are disclosed, an example method include: obtaining an image of an oocyte of a patient; extracting a plurality of morphological features from the image; and generating, based on the morphological features, a predicted value indicating a probability of the oocyte reaching a blastocyst development state.

### CROSS-REFERENCE TO RELATED APPLICATIONS

This application claims the benefits of and priority to U.S. Provisional Application No. 63/461,841 filed Apr. 25, 2023, the entire content of which is herein incorporated by reference.

### FIELD

This disclosure relates to systems and methods for predicting a quality or blastocyst development of an oocyte using an image of the oocyte. More specifically, this disclosure relates to non-invasive systems and methods for predicting blastocyst development of an oocyte from segmented images of the oocyte using machine learning models and feature engineering.

### BACKGROUND

Traditional methods of assessing quality of oocytes in fertility treatments often involve assessing the oocytes based on standard metrics of blastocyst development. However, the standard metrics can only act as a general guideline for assessing quality of the oocytes; reliably predicting the blastocyst development and the reproductive potential of oocytes for a patient however remains a difficulty in fertility clinics.

Therefore, there is a need to better evaluate qualities and reproductive potentials of oocytes that provides improved accuracy for clinicians and patients in making more sophisticated fertility related decisions.

### SUMMARY

Many further features and combinations thereof concerning embodiments described herein will appear to those skilled in the art following a reading of the present disclosure.

In one aspect, there is provided a computer-implemented system for predicting a quality of the oocyte, the system may include: a processor; and a memory coupled to the processor and storing processor-executable instructions that, when executed, configure the processor to: obtain an image of an oocyte of a patient; extract a plurality of morphological features from the image; and generate, based on the morphological features, a predicted value indicating a probability of the oocyte reaching a blastocyst development state.

In another aspect, a computer-implemented method for predicting a quality of an oocyte is disclosed, the method includes: obtaining an image of an oocyte of a patient; extracting a plurality of morphological features from the image; and generating, based on the morphological features, a predicted value indicating a probability of the oocyte reaching a blastocyst development state.

In some embodiments, the plurality of morphological features includes morphological features from one or more regions of: an ooplasm region, a perivitelline space (PVS) region, and a zona pellucida (ZP) region.

In some embodiments, the plurality of morphological features includes one or more of: a ratio of area, axis or diameter between the ooplasm region and the PVS region; a ratio of area, axis or diameter between the PVS region and the ZP region; and a diameter, a parameter or an area of the ooplasm region, the PVS region, or the ZP region.

In some embodiments, extracting the plurality of morphological features from the image includes: extracting the one or more regions from the image using an image segmentation model; determining a size of the one or more regions and the ratio between the one or more regions based on the extracted one or more regions; and computing a feature vector based on the size of the one or more regions and the ratio between the one or more regions.

In some embodiments, the feature vector may include features indicating an area of the one or more region, a measure of how round or irregular the shaped of one region is, or if a feature is found or otherwise.

In some embodiments, the image segmentation model comprises a neural network model, such as a region-based convolutional neural network (R-CNN) model, a U-Net convolutional neural network model, or a transformer model.

In some embodiments, the one or more regions comprise at least one of: an ooplasm region, a perivitelline space (PVS) region, and a zona pellucida (ZP) region.

In some embodiments, the system includes a prediction model used to generate the predicted value indicating the probability of the oocyte reaching the blastocyst development state.

In some embodiments, the feature vector is computed in accordance with a feature vector template pre-determined based on the prediction model.

In some embodiments, the prediction model includes a classifier model.

In yet another aspect, there is provided a non-transitory computer-readable medium having stored thereon machine interpretable instructions which, when executed by a processor, cause the processor to perform: obtaining an image of an oocyte of a patient; extracting a plurality of morphological features from the image; and generating, based on the morphological features, a predicted value indicating a probability of the oocyte reaching a blastocyst development state.

### BRIEF DESCRIPTION OF THE DRAWINGS

In the figures, embodiments are illustrated by way of example. It is to be expressly understood that the description and figures are only for the purpose of illustration and as an aid to understanding.

FIG. 1 is a schematic diagram of a computer-implemented system for predicting a quality of an oocyte, in accordance with an embodiment.

FIG. 2 illustrates a schematic diagram of an example neural network, in accordance with an embodiment.

FIG. 3 illustrates a schematic diagram of a computer-implemented feature engineering model, in accordance with an embodiment.

FIGS. 4A and 4B illustrates various example images of an oocyte.

FIG. 4C illustrates example segmented images of an oocyte showing example morphological characteristics.

FIG. 5 illustrates an example computer device for implementing a system for predicting a quality of an oocyte, in accordance with an embodiment.

FIG. 6 shows an example heat map for illustrating Shapley Additive explanation (SHAP) values various example features of an oocyte image.

FIG. 7 shows a table illustrating various morphological characteristics and measurements based on segmented images.

FIG. 8 illustrates an example process for predicting a quality of an oocyte as performed by the system in FIG. 1, in accordance with an embodiment.

### DETAILED DESCRIPTION

Disclosed herein includes system and methods implementing interpretable machine learning models for blastocyst development prediction of an oocyte, by leveraging oocyte image segmentation and feature engineering.

In some embodiments, as elaborated in detail below, an example computer-implemented system may be configured to extract one or more specific regions of the oocyte, including for example, without limitation, the ooplasm region, the perivitelline space (PVS) region, and the zona pellucida (ZP) region of the oocyte, which are correlated with various stages or states of blastocyst development of the oocyte.

By automatically generating segmented masks (which may also be referred to as segmented areas or regions) and generating a feature vector including salient features based on the morphological features of an oocyte, example embodiments of a system disclosed herein implement machine learning models that only use features related to the oocyte to generate a prediction regarding a probability of the oocyte reaching a blastocyst development state, resulting in increased accuracy and reliability in the prediction results.

In addition, the prediction results and the corresponding feature data can be displayed on a user interface on a device screen, in an easily interpretable and explainable form, by for example identifying regions within the oocyte on which the prediction was based, so that a clinician or user operating the system can easily understand, in a visual manner, how the prediction correlates to various features of the input oocyte image. This improves transparency of medical analysis performed by a machine learning system, inspires trust of users (e.g., clinicians and patients) in said machine learning system, and reduces barriers for users in general to adopt the system for use.

As a preliminary matter, some of the figures describe concepts in the context of one or more structural components, variously referred to as functionality, modules, features, elements, etc. The various components shown in the figures can be implemented in any manner, for example, by software, hardware (e.g., discrete logic components, etc.), firmware, and so on, or any combination of these implementations. In one embodiment, the various components may reflect the use of corresponding components in an actual implementation.

In other embodiments, any single component illustrated in the figures may be implemented by a number of actual components. The depiction of any two or more separate components in the figures may reflect different functions performed by a single actual component. The figures discussed below provide details regarding example systems that may be used to implement the disclosed functions.

Some concepts are described in form of steps of a process or method. In this form, certain operations are described as being performed in a certain order. Such implementations are example and non-limiting. Certain operations described herein can be grouped together and performed in a single operation, certain operations can be broken apart into plural component operations, and certain operations can be performed in an order that differs from that which is described herein, including a parallel manner of performing the operations. The operations can be implemented by software, hardware, firmware, manual processing, and the like, or any combination of these implementations. As used herein, hardware may include computer systems, discrete logic components, such as application specific integrated circuits (ASICs) and the like, as well as any combinations thereof.

As to terminology, the phrase “configured to” encompasses any way that any kind of functionality can be constructed to perform an identified operation. The functionality can be configured to perform an operation using, for instance, software, hardware, firmware and the like, or any combinations thereof.

As utilized herein, terms “component,” “system,” “client” and the like are intended to refer to a computer-related entity, either hardware, software (e.g., in execution), and/or firmware, or a combination thereof. For example, a component can be a process running on a processor, an object, an executable, a program, a function, a library, a subroutine, and/or a computer or a combination of software and hardware.

By way of illustration, both an application running on a server and the server can be a component. One or more components can reside within a process and a component can be localized on one computer and/or distributed between two or more computers. The term “processor” is generally understood to refer to a hardware component, such as a processing unit of a computer system.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any non-transitory computer-readable device, or media.

Non-transitory computer-readable storage media can include but are not limited to magnetic storage devices (e.g., hard disk, floppy disk, and magnetic strips, among others), optical disks (e.g., compact disk (CD), and digital versatile disk (DVD), among others), smart cards, and flash memory devices (e.g., card, stick, and key drive, among others). In contrast, computer-readable media generally (i.e., not necessarily storage media) may additionally include communication media such as transmission media for wireless signals and the like.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Example methods and systems disclosed herein allow for a single image analysis of the oocyte, and therefore does not require prolonged exposure. That is, example systems and methods disclosed herein may provide insight into likelihood of reaching a reproductive milestone in terms of blastocyst development based on one image of an oocyte.

Example application of artificial intelligence to assist with image analysis leads to an example automated and accurate oocyte classification system. The example classification and predictions may serve as a clinically valuable tool in both oocyte cryopreservation cases to help predict the potential outcomes of each oocyte, and in all failed in-vitro fertilization (IVF) cases to better understand the underlying etiology for the lack of success, for example, poor egg quality.

In an example embodiment, for a potential oocyte (or simply referred to as an oocyte) under consideration, an potential oocyte may be retrieved from an ovarian follicle. The potential oocyte may then be stripped and placed under a light microscope 160. An example camera mounted on an example light microscope 160 may capture an image of the stripped oocyte.

In some embodiments, the oocyte may be retrieved from a time-lapse incubator 170 as described in this disclosure.

In an example embodiment, an object of interest, that is, a potential oocyte or embryo to be evaluated may be identified in the captured image. A captured image may be cropped to isolate the object of interest. Utilizing developed parameters for both processing and analysing images as discussed in further detail below, artificial intelligence and various machine learning models may be utilized to determine the likelihood of potential successful outcomes with respect to the oocyte, by utilizing the cropped image focusing on the object of interest. In an example embodiment, an example validation score or prediction may be provided. Additionally, example supportive metrics may be provided, which may include chance of success and confidence in prediction, aiding a clinician in providing advice and guidance to potential patients on medical approaches.

While, the detailed description, focuses on determining quality of an oocyte in terms of blastocyst development, analogous example methods may be utilized for additional objects of interest such as embryos.

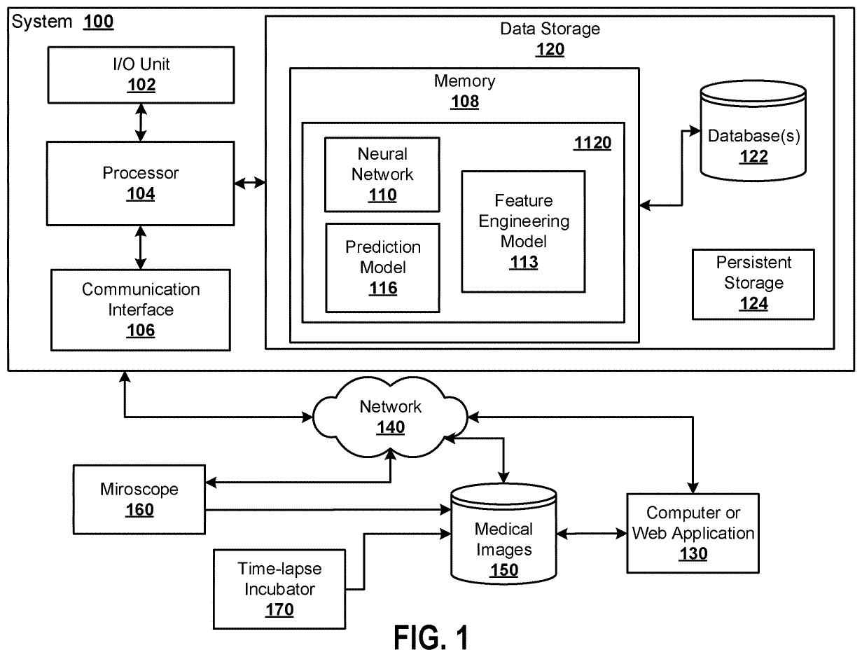

FIG. 1 is a schematic diagram of a computer-implemented system 100 for predicting oocyte a quality of an oocyte, in accordance with an embodiment. Determining quality of an oocyte may refer to a metric related to likelihood or probability of each of one or more of: a blastocyst development (development into a viable embryo), fertilization of the oocyte, euploidy status, implantation into the uterus, and clinical pregnancy. In some embodiments, quality of an oocyte may refer to a prediction regarding whether an oocyte will or will not reach a particular reproductive stage or milestone.

In some embodiments, input to system 100 may include an image 400. System 100 may perform image processing of the captured image data from image 400. Image processing of the captured image data may include, for example, cropping the captured image data so that the potential oocyte is the focus of the image and/or applying process of data augmentation. In an example embodiment, the captured image may be cropped with the oocyte in the center. In an example embodiment, an image of an object of interest (for example, a potential oocyte) may be at least 200 by 200 pixels after cropping.

In some embodiments, prior to extracting the morphological features using a feature engineering model 113, images may be processed by normalizing image brightness values, cropping irrelevant parts of an image, removing noise, or performing image sharpening.

Output from system 100 may include a predicted value, which may be a probability value representing a state or level of a blastocyst development of the oocyte, may be used as a metric for further determining or estimating a likelihood or potential of the oocyte to become fertilized or to lead to a clinical pregnancy. For example, the predictive value may be a probability value (e.g., 80%) of the oocyte reaching blastocyst on a certain date (e.g., day 5 or 6).

A machine learning application 1120 can maintain a neural network 110 to perform actions based on input data, which may include at least a single image of an oocyte. An example action may be image segmentation.

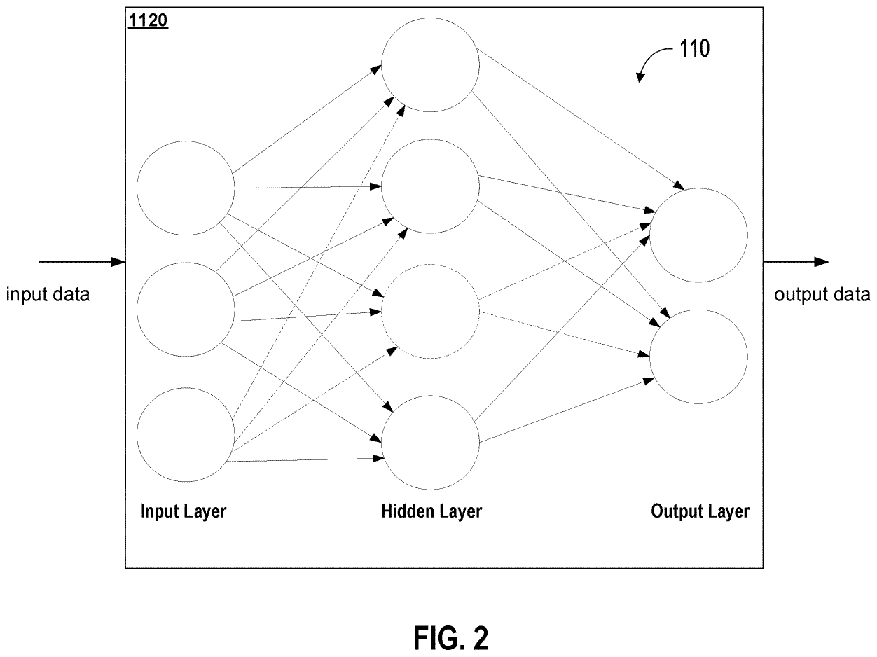

FIG. 2 shows an example neural network 110 being trained by a machine learning application 1120. The example neural network 110 can include an input layer, a hidden layer, and an output layer. The neural network 110 processes input data using its layers based on machine learning, for example. Once the neural network 110 has been trained, it generates output data reflective of its decisions to take particular actions (e.g., image segmentation) in response to particular input data. Input data may include one or more oocyte images, while output data may include a class label for each pixel within one or more regions or masks of the oocyte in the image, the class label indicating, for the respective pixel, the region, mask or morphological feature that this pixel belongs to. The output data may further include a bounding box offset for each pixel, and an object mask for each region in the input image 400. The output data may be used to generate one or more feature vectors that can be used as input data for a prediction model 116 for determining or estimating a likelihood or potential of an oocyte to become fertilized or to lead to clinical pregnancy.

In some embodiments, neural network 110 may be constructed as a region-based convolutional neural network (R-CNN) model, a U-Net convolutional neural network model, or a transformer model.

Referring back to FIG. 1, System 100 includes an I/O unit 102, a processor 104, a communication interface 106, and a data storage 120. I/O unit 102 enables system 100 to interconnect with one or more input devices, such as a keyboard, mouse, camera, a touch screen, a microscope 160, a time-lapse incubator 170, and/or with one or more output devices such as a display screen and a speaker.

Processor 104 executes instructions stored in memory 108 to implement aspects of processes described herein. For example, processor 104 may execute instructions in memory 108 to configure a data collection unit, neural network 110, machine learning application 1120, feature engineering model 113, prediction model 116, and other functions described herein.

Processor 104 can be, for example, various types of general-purpose microprocessor or microcontroller, a digital signal processing (DSP) processor, an integrated circuit, a field programmable gate array (FPGA), a reconfigurable processor, or any combination thereof.

Communication interface 106 enables system 100 to communicate with other components, to exchange data with other components (e.g., image database 150), to access and connect to network resources, to serve applications, and perform other computing applications by connecting to a network 140 (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi or WiMAX), SS7 signaling network, fixed line, local area network, wide area network, and others, including any combination of these.

Data storage 120 can include memory 108, databases 122, and persistent storage 124. Data storage 120 may be configured to store information associated with or created by the components in memory 108 and may also include machine executable instructions. Persistent storage 124 implements one or more of various types of storage technologies, such as solid state drives, hard disk drives, flash memory, and may be stored in various formats, such as relational databases, non-relational databases, flat files, spreadsheets, extended markup files, etc.

Data storage 120 stores a model for a machine learning neural network 110. The neural network 110 is trained and used by a machine learning application 1120 to generate one or more image segmentation masks based on one or more images, which may be transmitted from database 150.

Memory 108 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

System 100 may connect to a computer or web-based application 130 accessibly by a user device. The application 130 interacts with the system 100 to exchange data (including control commands) and generates visual elements for display at the user device. The visual elements can represent features from feature engineering model 113 and output generated by prediction model 116.

System 100 may be operable to register and authenticate users (using a login, unique identifier, and password for example) prior to providing access to applications, a local network, network resources, other networks and network security devices.

Processor 104 is configured to execute machine executable instructions (which may be stored in memory 108) to maintain a neural network 110, and to train neural network 110 of using one or more historical images which may be stored in database 122 or 150.

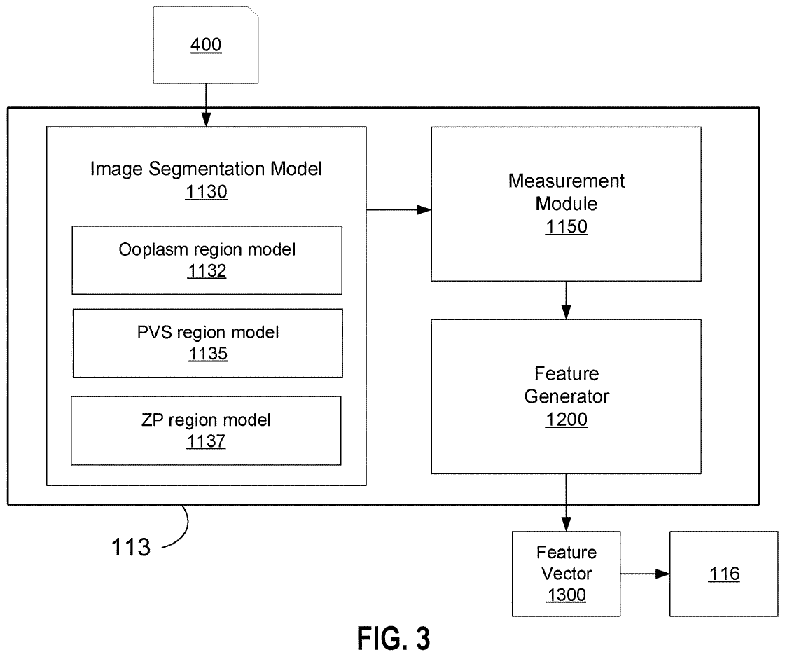

FIG. 3 illustrates a schematic diagram of a computer-implemented feature engineering model 113, in accordance with an embodiment.

An image 400 of an oocyte may be obtained, from for example a medical image database 150 and sent to the feature engineering model 113 for generating a feature vector 1300, which may be used by a prediction model 116 to compute a predicted value representing a quality of the oocyte in the image 400.

In an example embodiment, one or more images 400 of an oocyte may be captured with an image capturing device (not shown) attached to a light microscope 160. For example, images may be captured with Hoffman Modulation Contrast optics, at between 200 to 400× magnification or 20× to 40× objective. In some embodiments, inverted microscope, which is intracytoplasmic sperm injection (ICSI) compatible, and a C-Mount port or port with C-Mount adapter can be used for capturing the oocyte image.

In some embodiments, an oocyte image 400 may be retrieved from time-lapse incubator videos from a time-lapse incubator 170. The first couple of images of the time-lapse incubator video may represent a stripped mature oocyte within a few minutes after being injected with a single sperm via ICSI. The time-lapse incubator video traditionally may include sequential images from the oocyte to a blastocyst (day 5 or 6 of embryo development).

The oocyte images 400 may be stored in a database 150 connected to a network 140 and other network components.

In an example embodiment, images may be captured in grey scale. Exposure of an image capturing device (e.g., camera) may be adjusted in order to capture all details of the subject (for example, oocyte). For instance, the exposure may be adjusted so that no parts of a captured image may be completely black or completely white.

In an example embodiment, all captured images may go through a process of image or data augmentation, where the images may be transformed by one or more of scaling, rotating, flipping, and adjusting pixel values. In an example embodiment, data augmentation may allow for standardization of the all the captured images. In an example embodiment, standardizing all the captured images to user defined or automatically generated parameters may be valuable in terms of improving predictive accuracy.

In an example embodiment, captured images may have a 300× magnification. Resolution of the source image before cropping may be 3000 by 3000 pixels with a potential oocyte included within the image.

Image processing of the captured image data may comprise cropping the captured image data so that the potential oocyte is in the center and/or applying above-mentioned process of data augmentation. In an example embodiment, the captured image may be cropped with the object of interest in the center. In an example embodiment, an image of an object of interest (for example, a potential oocyte) may be at least 200 by 200 pixels after cropping. An image may be cropped around the oocyte, since the oocyte's shape is mostly round, the potential oocyte appears in the center of an image. In an example embodiment, a size of the cropped image may depend on the magnification of the lens on the microscope 160 and resolution of the camera.

Normalisation of pixel values may be applied.

The feature engineering model 113 may include three modules or components: an image segmentation model 1130, a measurement module 1150 and a feature generator 1200.

The image segmentation model 1130 may be used to generate segmented images based on the input image 400, each segmented image including a specific region of interest. For example, the image segmentation model 1130 may include one or more specific region models, such as, for example, an ooplasm region model 1132, a PVS region model 1135 and a ZP region model 1137.

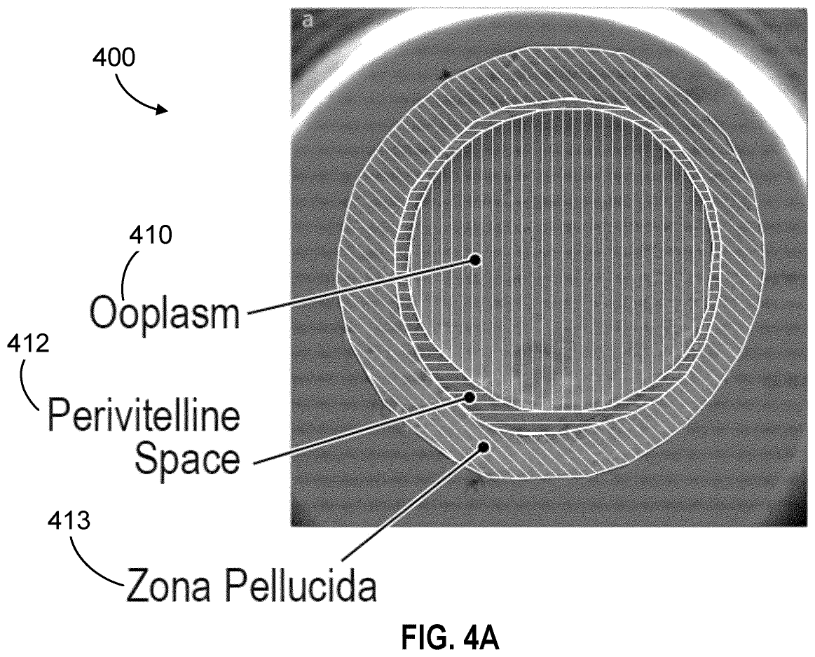

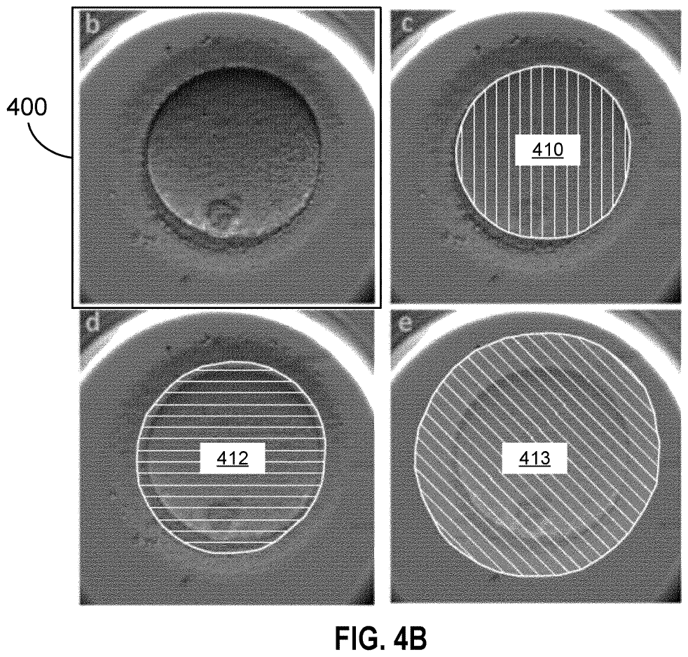

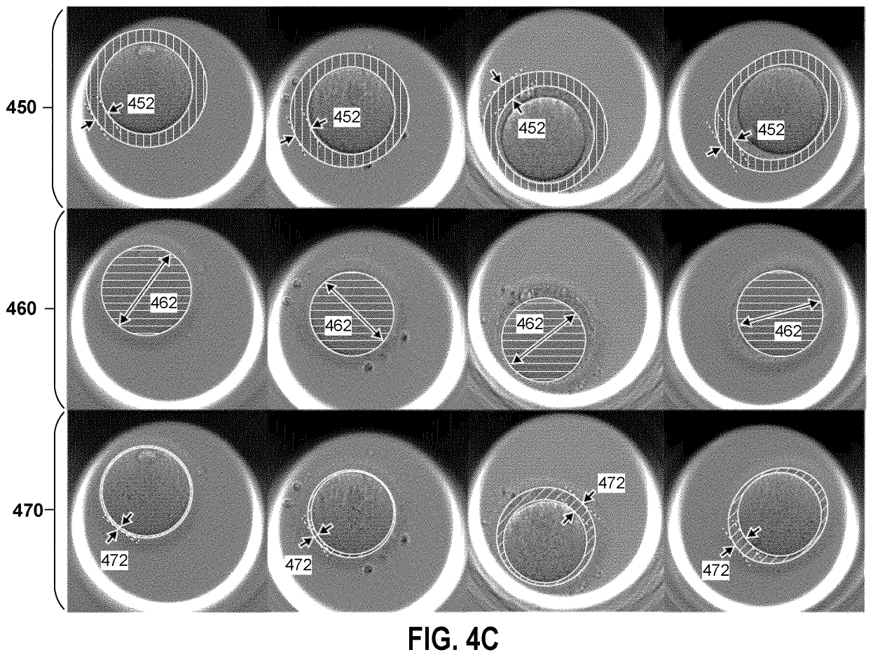

FIG. 4A illustrates an oocyte image with three regions, namely, ooplasm region 410, PVS region 412 and ZP region 413. FIG. 4B illustrates three segmented images, c, d and e, which are, respectively, c. segmented image with an ooplasm region 410, d. segmented image with a PVS region 412, and e. segmented image with a ZP region 413. The images c, d, and e can be output of the image segmentation model 1130. For instance, the ooplasm region model 1132 can be implemented to generate the segmented image with an ooplasm region 410, the PVS region model 1135 can be implemented to generate the segmented image with a PVS region 412, and the ZP region model 1137 can be implemented to generate the segmented image with ZP region 413.

In some embodiments, the segmentation of an image 400 to obtain the different regions or masks can be done algorithmically, instead of using a neural network model.

In some embodiments, the image segmentation model 1130 can analyze an image 400 and assign an index (or label) for each pixel describing which feature or region it belongs to.

The generated segmented images from the image segmentation model 1130 may be used to determine or extract a plurality of morphological features, which may include, for example, a size or ratio between different regions of the image 400, including for instance, a perimeter, area, major and minor axis length, aspect ratio, roundness, circularity, and solidity of each region, and their respective ratios. Based on the generated segmentation images (which may also be referred to as masks), a set of morphological characteristics, values or metrics for different oocyte regions 410, 412, 413, including the perimeter, area, major and minor axis length, aspect ratio, roundness, circularity, and solidity, can be determined, for example, by a measurement module 1150.

In some embodiments, relative features between two oocyte regions, such as the ratio between the ooplasm and PVS area, the ratio between the PVS and ZP area, ratio between the major or minor axis of ooplasm and PVS regions, as well as various combinations between any two pairs of segmented regions can be determined by the measurement module 1150.

System 100 including the feature engineering model 113 is configured to select salient or relevant features related to each segmentation region as generated by the image segmentation model 1130. Without using the feature engineering model 113, a routine machine learning model can spend computing resource on going through training or inference based on irrelevant features, such as noise or background in the images. The feature engineering model 113 is also more robust to changes in image quality as long as extracted features are a good representation of oocyte morphology.

In addition, system 100 as disclosed herein, can converge faster than traditional neural network (e.g., CNN) models without feature engineering model 113, as the dimension of the feature vector 1300 computed from the feature engineering model 113 is lower, and the inference speed of system 100 can therefore be much faster than traditional neural network models without feature engineering model 113.

In addition, system 100 can be configured for displaying tabular data for explaining features and their respective values in contributing to the final prediction result, on a display screen of a user device, as the features are readily available from the feature engineering model 113.

Whereas traditional CNN models without feature engineering require a large amount of training data to achieve high performance, system 100 performs better on smaller dataset with appropriate feature engineering. CNN models are also not sensitive to information such as shape or size of objects. Another benefit of the disclosed embodiments is that by engineering a feature vector for the prediction model, computational efficiency is greatly increased, as the prediction model 116 does not need to use computing resources for non-important features (noise in the signal), as the input feature vector to the prediction model defines exactly what model should pay attention to (i.e., the important features).

As mentioned above, the image segmentation model 1130 may be implemented using a neural network, such as a region-based convolutional neural network (R-CNN) model, a U-Net convolutional neural network model, or a transformer model. Training of the image segmentation model 1130 may be based on historical images of oocytes.

In some embodiments, a small number of samples of mature oocytes (500-2000) can be manually segmented by embryologists and used for training the image segmentation model 1130. Then images of oocytes, at inference time, can be automatically segmented, by the trained image segmentation model 1130, into different regions, including ooplasm, Perivitelline Space (PVS), and Zona Pellucida (ZP), using a deep learning algorithm trained based on embryologists' manual labels.

Oocytes images in the training data can be labeled based on blastocyst formation (e.g., positive if reached blastocyst on day 5 or 6, and negative if the oocyte did not reach blastocyst), or based on a quality of blastocyst on day 5 or 6 when the blastocyst is formed. Quality of blastocyst is typically correlated with higher Gardner score. When time-lapse images, such as from a time-lapse incubator 170, are available, oocytes can be labelled based on blastocyst development stages or states by a deep learning model trained on time-lapse data from the time-lapse images. Embryologists or machine learning algorithms can assess the labelled data quality, and data with artifacts or low image quality will be removed from the dataset.

In some embodiments, training data may be retrieved from a database, and may include a first set of images related to a plurality of oocytes and associated reproductive milestone data. In an example embodiment, the reproductive milestone data may refer to data related to fertilization, blastocyst development, pre-implantation genetic screening (PGS), euploidy status, implantation, and clinical pregnancy, wherein the data indicates whether the oocyte may have reached a particular reproductive milestone or not. In an example embodiment, a plurality of images associated with each of the plurality of oocytes may be included in the set of images. For example, utilizing time-lapse embryo incubators 170, a development of a particular oocyte may be monitored through fertilization on to blastocyst, with continuous images. Accordingly, the associated data may indicate whether each reproductive milestone for that particular oocyte was successful or otherwise. For example, fertilization, euploidy status or PGS results (if and when performed), and whether clinical pregnancy was successful of not. In example embodiments, the data may be parsed so that any influence on the data due to poor sperm quality is minimized.

In an example embodiment, preparing a clean and unbiased dataset may help develop a robust predictive model. A clean and unbiased data set may refer to both image quality and data linking, that is, an accurate record of images and reproductive outcomes. In an example embodiment, any images with undesirable qualities such as debris, shadowing, poor exposure, etc., may be removed from respective datasets.

The training data may include a first set of images of oocytes and their respective reproductive milestone outcomes. The outcome of each oocyte used in an training dataset may be accounted for, from fertilization to embryo transfer, thus producing a clean dataset. A large pool of oocyte images may be retrieved from time-lapse incubator videos. The first image of the time-lapse incubator video may represent a stripped mature oocyte within a few minutes after being injected with a single sperm (ICSI). The time-lapse incubator video traditionally may have sequential images from the oocyte to a blastocyst (day 5 or 6 of embryo development).

A second set of data may include captured single images of mature oocytes (after stripping, but before ICSI) using a camera mounted on a light microscope 160 and may be correlated with the image of the same oocyte (after ICSI) within the time-lapse incubator (first image of the time-lapse video). This second data set may be utilized to train a prediction model 116 that has been already trained on first images of the oocytes within the time-lapse incubator to recognize and predict outcomes on single image of a pre-ICSI mature oocyte under a light microscope 160.

FIG. 4C illustrates three rows 450, 460, 470 of segmented images, each row showing a respective morphological feature. Row 450 of segmented images shows an example morphological feature based on a measured difference 452 between a diameter of the first region and a diameter of the second region, where the first region may be the ZP region, and the second region may be the ooplasm region. The area, perimeter and diameter of each region may be computed by the measure module 1150 and used to determine a number of morphological features based on the size and shape of the two regions. For instance, in addition or instead of the measured difference 452 between the diameter of the first region and the diameter of the second region, the morphological features can also include a ratio of areas of the two regions, a difference in a major or minor axis of the two regions, or a ratio of perimeter of the two regions.

Row 460 of segmented images shows another example morphological feature, which may be area, perimeter or diameter 462 of the ooplasm region.

Row 470 segmented images shows a third example morphological feature, which may be based on a measured difference 472 between the diameter of the ZP region and the diameter the PVS region, as computed by the measure module 1150. The area, perimeter and diameter of each region may be computed by the measure module 1150 and used to determine a number of morphological features based on the size and shape of the two regions. For instance, in addition or instead of the measured difference 472 between the diameter of the first region and the diameter of the second region, the morphological features can also include a ratio of areas of the two regions, a difference in a major or minor axis of the two regions, or a ratio of perimeter of the two regions.

A number of morphological features may be selected by the feature generator 1200 based on an appropriate selection criteria in a feature vector template and used to generate a feature vector 1300. For example, SHAP importance values may be used to select one or more features for generating the feature vector 1300. The appropriate selection criteria may reduce redundancy, improve the performance of prediction models and gain insight in which features play main role in prediction.

In some embodiments, the feature generator 1200, based on a set of predetermined feature selection criteria specified by a feature vector definition, selects the appropriate features to generate the feature vectors. The feature vector definition, thereby the features, may be predetermined based on a specific task or action that is to be performed by prediction model 116.

For example, for a task of predicting a probability of the oocyte reaching a blastocyst development state at a future point in time, the feature vector 1300 may be a vector of n elements, namely, V=[v₁, v₂, v₃, . . . vₙ], where each element v_{i, i=1, 2, . . . n}, represents a selected feature value. For example, v₁ may be a ratio of area between the ooplasm region and the PVS region, v₂ may be a ratio of major ellipse axis between the ooplasm region and the PVS region, v₃ may be a ratio of major ellipse axis between the ooplasm and ZP region, and v₄ may be a roundness of the ooplasm region. The feature vector 1300 may further include clinical data such as: v₅ representing the age of the patient, and v₆ representing a number of mature oocytes from the patient.

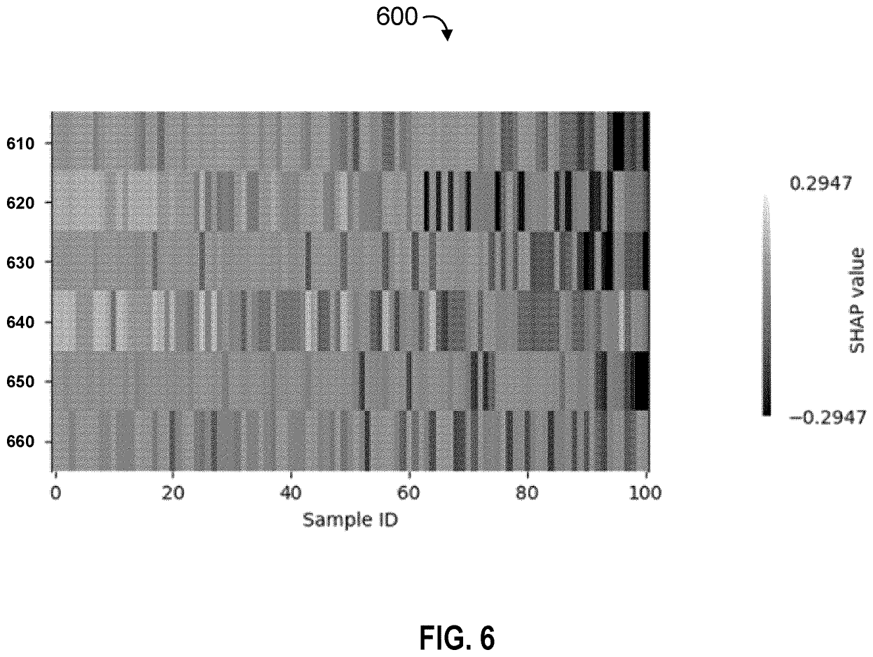

FIG. 6 shows an example heat map for illustrating Shapley Additive explanation (SHAP) values various example features of an oocyte image 400, rows 610 to 660 each represents, respectively:
the area ratio between the ooplasm region and PVS region 610;
age of the patient 620;
the major ellipse ratio between the ooplasm region and PVS region 630;
number of mature oocytes 640;
the major ellipse ratio between the ooplasm region and ZP region 650; and
roundness of the ooplasm region 660.

Each SHAP value of a respective feature indicates a local importance of said feature, which represents how much the feature has contributed, positively or negatively, to the prediction results across multiple samples of images. Generally speaking, features with highest positive SHAP values can be taken as those having the highest positive impact on the prediction model, such as prediction model 116. Therefore, SHAP values may be used for feature selection. In some embodiments, the global importance of features can be evaluated by a machine learning algorithm.

In some embodiments, SHAP values can be further implemented to provide an explainable machine learning system 100 for users of the system. For instance, using SHAP values, a number of most salient features can be plotted in a summary or chart, illustrating how much each respective feature has contributed to the prediction result of system 100. The summary or chart of features along with the prediction result may be rendered on a display screen of a user device.

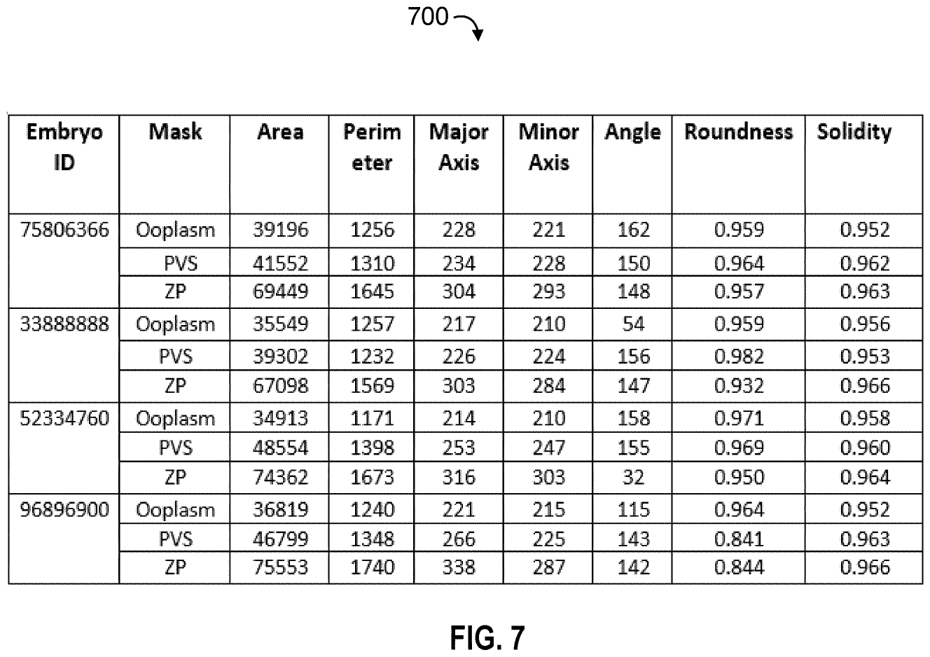

FIG. 7 shows a table 700 illustrating various morphological features, such as morphological characteristics and measurements, based on segmented images from an image segmentation model 1130. One of more of the morphological characteristics and measurements can be taken as one or more features by the feature generator 1120 to generate the feature vector 1130. The measurements shown in table 700 are based on pixel units, and images are resized to 500 by 500 pixies.

In some embodiments, a feature vector 1300 can include one or more morphological features of the image 400, as well as additional clinical data, such as clinical data about patient or specific treatment cycle, including for example age, body mass index (BMI), number of mature oocytes, and male factors.

In some embodiments, the feature vector 1300 may also include one or more of: information about other features of the cohort of oocytes, features extracted using Radiomics methodology, features relate to brightness, blurriness, and contrast of segmented images.

Referring back to FIG. 1, prediction model 116 can be a regression model, or a binary or multiple-classes classifier that has been trained with machine learning algorithms, such as random forests, support vectors machine, gradient boost machine, convolutional neural network (CNN) or transformer. The final prediction model 116 can be a single classifier, or the ensemble of multiple classifiers trained with different algorithms.

In some embodiments, deep learning models, such as a transformer can be used to train machine learning models with morphological features of the segmentation regions. The prediction model 116 can be a single model or an ensemble of multiple type of models. The ensemble can be majority votes or soft votes based on the prediction of multiple models. Equal or non-equal weights can be assigned to each model for ensemble.

In some embodiments, the prediction model 116 can, either as part of an ensemble of neural networks, or as a standalone neural network, include a CNN implemented to: extract target oocyte features from an input image 400 of an oocyte, compare target oocyte features with the extracted features, and output a result based on the comparison, such as described in U.S. Pat. Nos. 10,552,957B2 and 10,748,288B2, both of which are herein incorporated by reference in their respective entirety.

In some embodiments, prediction model 116 can include a classifier model. In some embodiments, prediction model 116 can include a Random Forest (RF) model and/or a Support Vector Machine (SVM) model.

In some embodiments, prediction model 116 can be trained using an iteration process, during which the machine learning engine and algorithm search for the optimal parameters that minimize the loss function and the best combination of hyper-parameters during training. Different loss functions, such as binary cross entropy loss, Hinge loss, can be used. An example machine learning model for prediction model 116 can be an optimizer, such as Adam, and SGD or its variant, can be applied to minimize the loss function during the training of prediction model 116.

For example, Bayesian optimization can be used for hyper-parameters searching. A training dataset can be split into train/validation/test sets for cross-validation.

During an inference stage of feature engineering model 113 and prediction model 116, segmentation images or masks based on oocyte morphological features are generated using the image segmentation model 1130. Each feature vector 1300 for an image 400 is computed based on the segmentation images or masks, clinical data and potentially images (if CNN model is used as prediction model 116). Prediction model 116 can map the feature vector 1300 into a final prediction, e.g., a predicted value. The predicted value may represent a probability of a blastocyst formation of an oocyte using morphological features of the oocyte. Statistical models can be applied, as a next step, to predict a probability of downstream events, such as fertilization, clinical pregnancy, and/or live birth based on the probability of each oocyte reaching a blastocyst. Models trained on extracted morphological features and using appropriate labels can be trained to other developmental outcomes like fertilization, blastocyst quality, euploidy status, implantation, and live birth.

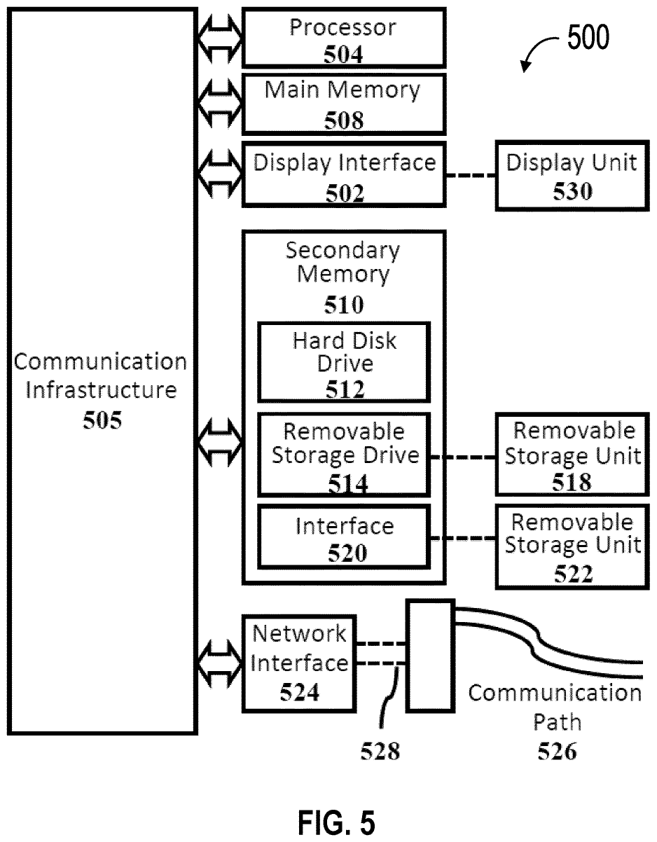

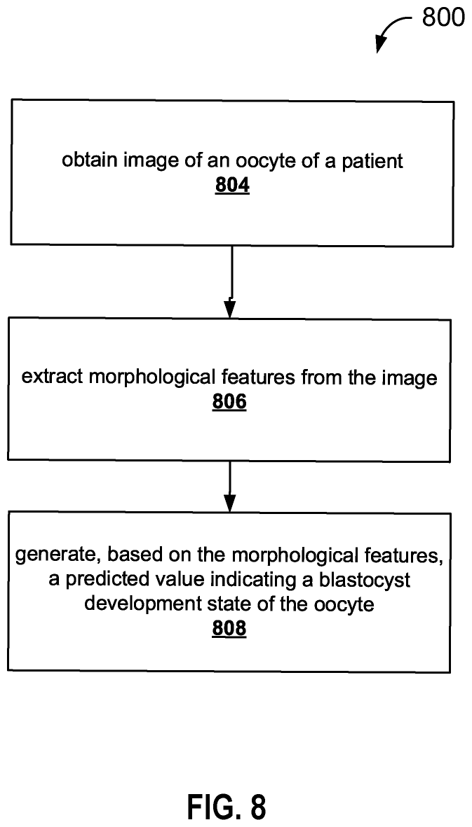

FIG. 8 illustrates a flowchart of a method 800 for machine learning architecture for predicting a quality of an oocyte, in accordance with embodiments of the present disclosure. The method 800 may be conducted by the processor 104 of the system 100 in FIG. 1 or system 500 in FIG. 5 described below. Processor-executable instructions may be stored in the memory 108 and may be associated with the machine learning application 1120 or other processor-executable applications. The method 400 may include operations such as data retrievals, data manipulations, data storage, or other operations, and may include computer-executable operations.

At operation 804, the processor may obtain or receive an image 400 of an oocyte of a patient. For example, an image of a patient's sample oocyte may be taken to determine its likelihood of reaching each of the reproductive milestones. The patient's sample oocyte may also be referred to as a potential oocyte, that is, a potential oocyte which may be used in a reproductive process. In an example embodiment, a clinician or a physician may be tasked with capturing an image of a potential oocyte. The captured image may be uploaded to a cloud environment or transferred to a computing system using a network or other methods. In an example embodiment, an image of a potential oocyte may be captured utilizing an image capturing device, such as a camera, attached to a light microscope 160.

Operation 804 may include, in some embodiments, stripping a potential oocyte. In an example embodiment, stripping may refer to separating the potential oocyte from other surrounding cells. This may be done under an example high-power microscope, such as an ICSI microscope. In an example embodiment, before stripping the potential oocyte, it may be retrieved from an ovarian follicle.

Operation 804 may include, in some embodiments, utilizing an image capturing device to capture image data associated with the potential oocyte. In an example embodiment, the image of the potential oocyte may be captured after stripping and immediately pre-ICSI, pre-freezing, or post-freezing. The oocyte may be thawed post-freezing and an image of the oocyte may be taken using the image capturing device, such as a camera attached to a microscope 160.

In an example embodiment, settings and/or conditions for capturing an image of a potential oocyte may be the same as the previously captured images referred to in training of the image segmentation model 1130, captured from a light microscope 160. For example, a camera attached to an example high-power microscope 160 may be used to capture the image of a potential oocyte. For example, images may be captured with Hoffman Modulation Contrast optics, at between 200 to 400× magnification. In an example embodiment, images may be captured in grey scale. In an example embodiment, exposure of an example camera may be adjusted in order to capture all details of the subject to be captured. In an example embodiment, the exposure may be adjusted so that all parts of the potential object to be evaluated are clear.

Operation 804 may include, in some embodiments, image processing of the captured image data. Image processing of the captured image data may include cropping the captured image data so that the potential oocyte is in the center and/or applying above-mentioned process of data augmentation. In an example embodiment, the captured image may be cropped with the object of interest in the center. In an example embodiment, an image of an object of interest (for example, a potential oocyte) may be at least 200 by 200 pixels after cropping. An image may be cropped around the oocyte, since the oocyte's shape is mostly round, the potential oocyte appears in the center of an image. In an example embodiment, a size of the cropped image may depend on the magnification of the lens on the microscope 160 and resolution of the camera.

In an example embodiment, capturing an image of an object of interest, such as a potential oocyte may be conducted in similar manner and specification as capturing of the images used for training data sets. In an example embodiment, this may allow for more efficiency and accuracy in predictive accuracy of example systems.

Operation 806 may include extracting a plurality of morphological features from the image 400. The plurality of morphological features may be used to compute a feature vector 1300 based on at least a size of one or more regions and a ratio between the one or more regions, which may include, for example, an ooplasm region, a perivitelline space (PVS) region, and a zona pellucida (ZP) region.

For example, an image segmentation model 1130 may extract the one or more regions from the image using an image segmentation model; a measurement module 1150 may determine morphological features or values, including for example, the size of the one or more regions and the ratio between the one or more regions based on the extracted one or more regions; and a feature generator 1200 may compute the feature vector 1300 based on the size of the one or more regions and the ratio between the one or more regions.

The feature vector 1300 may include one or more morphological features, including for example, an area ratio between the ooplasm region and PVS region, a major ellipse ratio between the ooplasm region and PVS region, a major ellipse ratio between the ooplasm region and ZP region; roundness of the ooplasm region, and an area, perimeter or diameter of each of the segmented regions.

The feature vector 1300 may include additional feature data based on clinical data, including for example, a number of mature oocytes of the patient, an age of the patient, and so on.

Operation 808 may include generating, for example using a prediction model 116, based on the feature vector 1300 including the morphological features, a predicted value indicating a probability of the oocyte a blastocyst development state from the input image 400. For instance, the predicted value may indicate a likelihood of reaching each of the reproductive milestones of the potential oocyte based on the feature vector 1300. Specifically, a likelihood in terms of percentage, a ranking, or a numerical value may be calculated for fertilization, blastocyst development, euploidy status, PGS (pre-implantation genetic screening), implantation, and clinical pregnancy.

In some embodiments, the prediction model 116 may compare target oocyte features from training data with the extracted features. The prediction model 116 may output a result based on the comparison. For example, if the potential oocyte contains features which correlate with likelihood of each reproductive milestone (described above) or lack thereof, the results may be output.

In an example embodiment, analogous methods as discussed above with respect to method 800 with regards to oocyte quality and predicting likelihood of fertilization, blastocyst development, euploidy status, PGS, implantation and clinical pregnancy may be used for numerous other processes. For example, analogous example methods may be utilized for sperm assessment (that is, select the best potential sperm to fertilize an egg with), embryo assessment, and uterine cavity assessment.

In some embodiments, a U-Net model capable of accurately segmenting or masking the oocyte image is implemented as an image segmentation model 1130. In experiments using the U-Net model for segmenting the oocyte image, experimental data show that the average intersection over union (IOU) scores are 97.98%±0.10% for ooplasm region, 96.60%±0.15% for PVS region, and 97.29%±0.16% for ZP region, indicating a high percentage of overlap between ground truth training data and the segmentation results from the U-net model, which means that the U-net model is very effective in extracting specific regions from the oocyte image for feature engineering.

In experiments, a number of features including for example, roundness, major and minor axes, measurements from the extracted or segmented regions, have been determined from the segmented images based on a given oocyte image 400. A classification model (e.g., a LightGBM classifier model), trained based on a dataset of over 22,000 historical images, is implemented within prediction model 116 to process said features and additional clinical features (e.g. an age of the oocyte) to generate a prediction on blastocyst development of the oocyte. Mean absolute Shapley (e.g., SHAP) values across all samples in the dataset are used to determine global feature importance and illustrated herein (see e.g., FIG. 6).

On a test set of 6133 segmented images from two fertility clinics, the blastocyst prediction model displayed an AUC (area under Receiver Operator Characteristic curve) of 0.63 for Fertility Clinic #1 and an AUC of 0.64 for Fertility Clinic #2. The features demonstrated to be significantly prognostic of oocyte quality (as defined by blastocyst development) were the woman age (mean Shapley value of 0.1), the area ratio and major ellipse ratio between the ooplasm and PVS (both with mean Shapley values of 0.1), number of mature oocytes (mean 0.09), the major ellipse ratio between the ooplasm and ZP (mean 0.08), and ooplasm roundness (mean 0.06).

As disclosed herein, the image segmentation model 1130 can accurately segment the three principal regions of the oocyte, and the segmented images can then be used to generate a feature vector including morphological features of the oocyte for input to the prediction model 116. The prediction model 116, with an AUC of 0.63 and 0.64 on two fertility clinic test sets, demonstrates favorable performance in determining developmental competence of an oocyte, as defined by blastocyst development.

FIG. 5 illustrates another example computer system 500 in which an embodiment of the present disclosure, or portions thereof, may be implemented as computer-readable code, consistent with example embodiments of the present disclosure. For example, computer system 500 may include hardware, software, firmware, tangible computer readable media having instructions stored thereon, or a combination thereof and may be implemented in one or more computer systems or other processing systems. Hardware, software, or any combination of such may embody any of the modules and components utilized with respect to the process described in FIG. 8.

If programmable logic is used, such logic may execute on a commercially available processing platform or a special purpose device. One of ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 is connected to a communication infrastructure 505, for example, a bus, message queue, network, or multi-core message-passing scheme.

Computer system 500 also includes a main memory 508, for example, random access memory (RAM), and may also include a secondary memory 510. Secondary memory 510 may include, for example, a hard disk drive 512, removable storage drive 514. Removable storage drive 514 may comprise a floppy disk drive, a magnetic tape drive, an optical disk drive, a flash memory, or the like. The removable storage drive 514 reads from and/or writes to a removable storage unit 518 in a well-known manner. Removable storage unit 518 may comprise a floppy disk, magnetic tape, optical disk, etc., which is read by and written to by removable storage drive 514. As will be appreciated by persons skilled in the relevant art, removable storage unit 518 includes a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from the removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement the present invention as discussed herein. In particular, the computer programs, when executed, enable processor device 504 to implement the processes of the present invention, such as the operations in the method 800 illustrated by FIG. 8 discussed above.

Accordingly, such computer programs represent controllers of the computer system 500. Where the invention is implemented using software, the software may be stored in a computer program product and loaded into computer system 500 using removable storage drive 514, interface 520, and hard disk drive 512, or communications interface 524.

Embodiments of the invention also may be directed to computer program products comprising software stored on any computer useable medium. Such software, when executed in one or more data processing device, causes a data processing device(s) to operate as described herein. An embodiment of the invention employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described example embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic; e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be example only.

1. A computer-implemented system for predicting a quality of an oocyte, comprising:
a processor; and
a memory coupled to the processor and storing processor-executable instructions that, when executed, configure the processor to:
obtain an image of an oocyte of a patient;
extract a plurality of morphological features from the image; and
generate, based on the morphological features, a predicted value indicating a probability of the oocyte reaching a blastocyst development state.

2. The system of claim 1, wherein the plurality of morphological features comprises morphological features from one or more regions of: an ooplasm region, a perivitelline space (PVS) region, and a zona pellucida (ZP) region.

3. The system of claim 2, wherein the plurality of morphological features comprises one or more of: a ratio of area, axis or diameter between the ooplasm region and the PVS region; a ratio of area, axis or diameter between the PVS region and the ZP region; and a diameter, a parameter or an area of the ooplasm region, the PVS region, or the ZP region.

4. The system of claim 2, wherein extracting the plurality of morphological features from the image comprises:
extracting the one or more regions from the image using an image segmentation model;
determining a size of the one or more regions and a ratio between the one or more regions based on the extracted one or more regions; and
computing a feature vector based on the size of the one or more regions and the ratio between the one or more regions.

5. The system of claim 4, wherein the image segmentation model comprises a neural network model.

6. The system of claim 4, wherein the image segmentation model comprises a region-based convolutional neural network (R-CNN) model, a U-Net convolutional neural network model, or a transformer model.

7. The system of claim 4, further comprising a prediction model to generate the predicted value indicating the probability of the oocyte reaching the blastocyst development state.

8. The system of claim 7, wherein the feature vector is computed in accordance with a feature vector template pre-determined based on the prediction model.

9. The system of claim 8, wherein the prediction model comprises a classifier model.

10. A computer-implemented method for predicting a quality of an oocyte, the method comprising:
obtaining an image of an oocyte of a patient;
extracting a plurality of morphological features from the image; and
generating, based on the morphological features, a predicted value indicating a probability of the oocyte reaching a blastocyst development state.

11. The method of claim 10, wherein the plurality of morphological features comprises morphological features from one or more regions of: an ooplasm region, a perivitelline space (PVS) region, and a zona pellucida (ZP) region.

12. The method of claim 11, wherein the plurality of morphological features comprises one or more of: a ratio of area, axis or diameter between the ooplasm region and the PVS region; a ratio of area, axis or diameter between the PVS region and the ZP region; and a diameter, a parameter or an area of the ooplasm region, the PVS region, or the ZP region.

13. The method of claim 11, wherein extracting the plurality of morphological features from the image comprises:
extracting the one or more regions from the image using an image segmentation model;
determining a size of the one or more regions and a ratio between the one or more regions based on the extracted one or more regions; and
computing the feature vector based on the size of the one or more regions and the ratio between the one or more regions.

14. The method of claim 13, wherein the image segmentation model comprises a neural network model.

15. The method of claim 13, wherein a prediction model is used to generate the predicted value indicating the probability of the oocyte reaching the blastocyst development state.

16. The method of claim 13, wherein the feature vector is computed in accordance with a feature vector template pre-determined based on the prediction model.

17. The method of claim 16, wherein feature vector is computed in accordance with a feature vector template pre-determined based on the prediction model.

18. The method of claim 17, wherein the prediction model is a classifier model.

19. A non-transitory computer-readable medium having stored thereon machine interpretable instructions which, when executed by a processor, cause the processor to perform:
obtaining an image of an oocyte of a patient;
extracting a plurality of morphological features from the image; and
generating, based on the morphological features, a predicted value indicating a probability of the oocyte reaching a blastocyst development state.
